(19) United States
(12) Patent Application Publication
(10) Pub. No.: US 2020/0141948 A1
(43) Pub. Date: May 7, 2020

(54) METHOD AND KIT FOR DIAGNOSIS OF MUSCLE WEAKNESS-RELATED DISEASES USING BLOOD BIOMARKER

(71) Applicant: KOREA RESEARCH INSTITUTE OF BIOSCIENCE AND BIOTECHNOLOGY, Daejeon (KR)

(72) Inventors: Ki-Sun KWON, Daejeon (KR); Ju Yeon KWAK, Daejeon (KR); Seung-Min LEE, Daejeon (KR); Jeong Yi CHOI, Daejeon (KR); Kwang-Pyo LEE, Daejeon (KR); Eun-Soo KWON, Daejeon (KR)

(73) Assignee: KOREA RESEARCH INSTITUTE OF BIOSCIENCE AND BIOTECHNOLOGY, Daejeon (KR)

(21) Appl. No.: 16/625,112

(22) PCT Filed: Jun. 19, 2018
(86) PCT No.: PCT/KR2018/006928

§ 371 Date: Dec. 20, 2019

(30) Foreign Application Priority Data
Jun. 21, 2017 (KR) 10-2017-0078488

(51) Int. Cl. G01N 33/68; G01N 33/543

(52) U.S. Cl. CPC G01N 33/6893; G01N 33/54306; G01N 2333/65; G01N 2800/2878; G01N 2333/5412; G01N 2800/10

(57) ABSTRACT

The present invention relates to a composition and a kit for diagnosis of muscle weakness-related disease, which comprises agents for measuring the expression levels of gelsolin and tetranectin, and to a method of diagnosing muscle weakness-related disease by using the same. The composition, kit and method for diagnosis of muscle weakness-related disease according to the present invention make it possible to diagnose muscle weakness-related disease in an easy and rapid manner by molecular diagnosis, thereby systemically managing the muscle weakness-related disease while increasing therapeutic efficacy against the muscle weakness-related disease.

### TECHNICAL FIELD

The present invention relates to a composition and a kit for diagnosis of muscle weakness-related disease, which comprises an agent for measuring the expression level of gelsolin or tetranectin, and to a method of diagnosing muscle weakness-related disease by using the same.

### BACKGROUND ART

Everyone's muscle mass is reduced by about 10-15% at 50-70 years old and by at 15 least 30% at 70-80 years old, which causes a decrease in muscle strength and function. In particular, sarcopenia refers to a reduction in muscle strength with a decrease in skeletal muscle mass due to aging. Not only the decrease in muscle mass, which is the most important characteristic of sarcopenia, but also changes in the type of muscle fibers are observed. The thicknesses of type 1 and type 2 muscle fibers decrease at similar rates, 20 whereas under sarcopenia, the thickness of type 2 muscle fibers does not change significantly, but the thickness of type 1 muscle fibers noticeably decreases. It has been reported that this sarcopenia causes senescence and dysfunctions among the elderly (RoubenoffR, Can. J. Appl. Physiol. 26, 78-89, 2001).

Diseases that cause muscle weakness include: sarcopenia which progresses with aging muscular atrophy which is caused by an imbalance in protein metabolism and a decrease in muscle use; muscle dystrophy; cachexia; and acardiotrophy, which progresses with starvation, debilitating diseases (e.g., cancer, etc.), and aging.

Muscular atrophy or muscle wasting can be defined as the wasting or loss of muscle tissue that occurs due to a disease of muscle itself damage to the nerves that control muscles, or the disuse of muscles. The common cause may be the so-called “disuse atrophy” that occurs due to the disuse of muscles. Namely, in the case of a person whose social activity is decreasing, the muscle tone itself decreases, leading to progressive atrophy. This type of atrophy can be recovered to some extent by active exercise.

Unlike this, if a person has to lie in bed, serious muscle wasting will occur. In addition, people living in places without gravity (absence of frictional force) also show symptoms of decreased muscle strength due to decreased calcium and muscle strength.

In addition, the causes other than the disuse can be roughly divided into two types.

First, muscle atrophy caused by damage to the nerves that control muscles includes the following diseases.

Amyotrophic lateral sclerosis (ALS; also called Lou Gehrig's disease) is a disease in which nerve conduction to the muscle does not occur due to abnormalities in the myelin sheath surrounding motor nerves that move the muscles, resulting in loss of muscle motility, which results in muscle atrophy. Guillain-Barre syndrome (acute inflammatory demyelinating polyneuropathy) is a disease that occurs in children due to structural defects in the myelin sheath, like ALS. It is a disease that begins from leg muscles and gradually climbs up to the upper body muscles, eventually paralyzing the respiratory muscles (diaphragm muscles), resulting in death due to dyspnea.

Second, muscle atrophy caused by a disease of muscle itself includes the following diseases.

Myasthenia gravis is a disease that causes abnormalities in transmission of the neurotransmitters acetylcholine which transmits electrical signals to muscle fibers. This disease occurs because nerve impulses are not transmitted to muscles due to the congenital or acquired absence of acetylcholine receptors in postsynaptic muscle fibers or the decrease in number of receptors caused by antibody attack.

Muscular dystrophy is a genetic disease that occurs in the muscle itself without damage to the central nervous system or peripheral nervous system. This disease can be diagnosed within only one year or one and a half years after birth, but appears at the age of 2 to 4 years in most cases, and may also occur at mature ages in some cases. Muscular dystrophy refers to a collection of more than 30 genetic diseases that cause progressive weakening and degeneration of skeletal muscles which are used during autonomous exercise. In all types of muscular dystrophy, the muscles progressively degenerate and weaken, and eventually many patients lose their ability to walk.

This muscular dystrophy is divided into nine major groups. Specifically, it is divided, according to the extent and distribution of muscle wakness, age at onset, the speed of progression, the severity of symptos, family history and the like, into Duchenne muscular dystrophy, Becker muscular dystrophy, limb-girdle muscular dystrophy, Emery-Dreifuss muscular dystrophy, facioscapulohumeral muscular dystrophy, myotonic muscular dystrophy, oculopharyngeal muscular dystrophy, distal muscular dystrophy, and congenital muscular dystrophy.

Cachexia or wasting syndrome is characterized by weight loss, muscle atrophy fatigue, weakness, reduced appetite, and the like. In the case of cachexia, weight loss is not restored even by ingestion of nutrients. Cachexia may occur in patients with diseases such as cancer, AIDS, celiac disease, chronic obstructive pulmonary disease (COPD), multiple sclerosis, congestive heart failure, tuberculosis, familial amyloid polyneuropathy, mercury poisoning (acrodynia), and hormone deficiency. The occurrence of cachexia in these patients can be regarded as an increase in the severity of the disease in the patients, and patients suffering from cachexia may have increased immobility due to increased weakness and fatigue, and they may not respond effectively to conventional treatments.

Sarcopenia is also a pathological symptom of cachexia Sarcopenia is caused by various factors, but research on each of the factors is still insufficient. It is induced by a decrease or neurological change in growth hormones, a change in physiological activity, a change in metabolism, an increase in the amounts of sex hormones, fats or catabolic cytokines, and a changed balance between the synthesis and differentiation of proteins (Roubenoff R and Hughes V. A, J. Gerontol. A. Biol. Sci. Med. Sci. 55, M716-M724, 2000).

In addition, sarcopenia is increasing rapidly due to the aging of the population, and was recently coded in ICD-10-CM (Clinical Modification) and assigned disease code number M62.84, and thus its importance has grown. However, there has been no development of a test tool for diagnosing muscle aging that may occur in the entire elderly population. At present, physical tests (hand grip strength, walking speed, etc.) and dual-energy X-ray absorptiometry (DEXA) radiography (or CT) are used as test tools. However, the above tests have problems in that they are very inconvenient, cause radiation hazards, and are uneconomical.

Therefore, it is urgent to research and develop methods capable of diagnosing muscle weakness-related diseases, including sarcopenia, in a simple manner by molecular diagnostic tools' instead of the conventional methods as described above.

### PRIOR ART DOCUMENTS

### Patent Art Documents

KR 10-2015-0131556 (Nov. 25, 2005);

KR 10-2011-0001068 (Jan. 6, 2011).

### DISCLOSURE OF INVENTION

### Technical Problem

The present invention is directed to a composition for diagnosis of muscle weakness-related disease, which comprises an agent for measuring the expression level of tetranectin protein.

The present invention is also directed to a composition for diagnosis of muscle weakness-related disease, which comprises an agent for measuring the expression level of gelsolin protein.

The present invention is also directed to a composition for diagnosis of muscle weakness-related disease, which comprises agents for measuring the expression levels of tetranectin and gelsolin proteins.

The present invention is also directed to a kit for diagnosis of muscle weakness-related disease, which comprises a composition for diagnosis of muscle weakness-related disease, which comprises agents for measuring the expression levels of tetranectin and gelsolin proteins.

The present invention is also directed to a method for providing information for diagnosis of muscle weakness-related disease, the method comprising the steps of: (a) measuring the expression levels of tetranectin and gelsolin proteins in a biological sample obtained from a subject; and (b) determining that the subject has muscle weakness-related disease, when the expression levels of tetranectin and gelsolin proteins are higher than those in a normal group.

The present invention is also directed to a method for providing information for diagnosis of muscle weakness-related disease, the method comprising the steps of: (a) measuring the expression levels of tetranectin, gelsolin, and any one or more proteins selected from the group consisting of macrophage migration inhibitory factor (MIF), interleukin-6 (IL-6), SPARC (secreted protein acidic and rich in cysteine) and insulin-like growth factor-1 (IGF-1), in a biological sample obtained from a subject; (b) calculating a risk score based on the expression levels measured in step (a); and (c) comparing the calculated risk score with a reference level, and determining that the subject has muscle weakness-related disease, when the risk score is equal to or higher than the reference level.

### Solution to Problem

The present inventors have made extensive efforts to diagnose muscle weakness-related disease by molecular diagnostic technology instead of methods such as physical tests or radiography, and as a result, have found that muscle weakness-related disease can be diagnosed by measuring the concentration of gelsolin or tetranectin protein in blood and calculating a risk score based on the measured protein concentration, thereby completing the present invention.

The present invention is intended to provide a composition for diagnosis of muscle weakness-related disease, which comprises an agent for measuring the expression level of tetranectin protein.

Tetranectin is a plasma protein belonging to the C-type lectin domain family, encoded by the CLEC3B gene, and is composed of four polypeptide chains, each consisting of 181 amino acids (gene sequence: NM_001308394; amino acid sequence: NP_001295323).

The use of the composition for diagnosis of muscle weakness-related disease according to the present invention makes it possible to diagnose muscle weakness-related disease with high accuracy by measuring a change in the expression level of tetranectin.

The present invention is also intended to provide a composition for diagnosis of muscle weakness-related disease, which comprises an agent for measuring the expression level of gelsolin protein.

Gelsolin is an actin-binding protein composed of six subdomains and having a molecular weight of about 82 kDa (gene sequence: NM_000177; amino acid sequence: NP_000168).

The use of the composition for diagnosis of muscle weakness-related disease according to the present invention makes it possible to diagnose muscle weakness-related to disease with high accuracy by measuring a change in the expression level of gelsolin.

The present invention is also intended to provide a composition for diagnosis of muscle weakness-related disease, which comprises agents for measuring the expression levels of tetranectin and gelsolin proteins. The use of the composition for diagnosis of muscle weakness-related disease according to the present invention makes it possible to diagnose muscle weakness-related disease with a significant higher accuracy than single markers of tetranectin and gelsolin by measuring changes in the expression levels of tetranectin and gelsolin.

As used herein, the term “muscle weakness-related disease” refers to a condition in which the strength of one or more muscles is reduced. The muscle weakness may be limited to any one muscle, one side of the body, the upper or lower extremities, or the like, and may also appear throughout the whole body. In addition, subjective muscle weakness symptoms, including muscle fatigue pain, can be quantified in an objective way through physical examinations.

Muscle weakness-related disease in the present invention refers to all diseases that can be caused by muscle weakness. For example, the muscle weakness-related disease may be any one or more selected from the group consisting of sarcopenia, muscular atrophy, muscular dystrophy, cachexia, and acardiotrophy, but is not limited thereto. According to one embodiment of the present invention, the muscle weakness-related disease may be sarcopenia or muscular atrophy.

Sarcopenia in the present invention refers to a decrease in muscle strength with a decrease in skeletal muscle mass due to aging. For example, sarcopenia means disorders caused by aging, such as a decrease in muscle mass, a change in the type of muscle fibers, and a decrease in the thickness of muscle fibers.

Muscular atrophy in the present invention refers to a disease in which the muscles of the limbs continue to shrink almost symmetrically, and which is the wasting or loss of muscle tissue that occurs due to a disease of muscle itself damage to the nerves that control muscles, or the disuse of muscles. Specifically, muscular atrophy includes disuse atrophy of muscles, amyotrophic lateral sclerosis (ALS), spinal progressive muscular atrophy (SPMA), Guillain-Barre syndrome, myathenia gravis, and the like.

Muscular dystrophy in the present invention refers to a genetic disease that occurs in the muscle itself without damage to the central nervous system or peripheral nervous system. This disease can be diagnosed within only one year or one and a half years after birth, but appears at the age of 2 to 4 years in most cases, and may also occur at mature ages in some cases. Muscular dystrophy refers to a collection of more than 30 genetic diseases that cause progressive weakening and degeneration of skeletal muscles which are used during autonomous exercise.

Cachexia in the present invention refers to a high degree of general weakness which is characterized by weight loss, muscle atrophy, fatigue, weakness, reduced appetite, and the like and in which weight loss is not restored even by ingestion of nutrients.

Acardiotrophy in the present invention refers to the atrophy of the heart by external or internal factors. Due to starvation, debilitating diseases, or senility, myocardial fibers become skinner and thinner, leading to brown atrophy of the heart, which results in a reduction in adipose tissue.

The term “diagnosis” as used herein includes determination of a subject's susceptibility to a particular disease or disorder, determination as to whether a subject is presently affected by a particular disease or disorder, prognosis of a subject affected by a particular disease or disorder, and use of therametrics (e.g., monitoring a subject's condition to provide information as to the effect or efficacy of therapy). For the purpose of the present invention, diagnosis includes determining whether muscle weakness-related to disease would develop, the possibility (risk) of developing the disease, the degree of progression of the disease, and the like.

As used herein, the term “biomarker”, “marker” or “diagnostic marker” refers to a marker capable of distinguishing between normal and pathological conditions or predicting and objectively measuring therapeutic responses. In particular, regarding the muscle weakness-related disease in the present invention, the term means a marker whose protein expression level or gene expression level significantly increases or decreases in an individual having muscle weakness-related disease or being at risk of developing muscle weakness-related disease, compared to a normal control (an individual having no muscle weakness-related disease).

As used herein, “measuring the expression level of protein” means a process of determining the presence and expression level of a muscle weakness-related disease diagnostic marker (protein) or a gene encoding the same in a biological sample in order to diagnose muscle weakness-related disease.

Agents for measuring the expression of protein as described above include antibodies, substrates, peptide aptamers, receptors interacting specifically with the marker, ligands, cofactors or the like. Specifically, the agents include antibodies specific for proteins encoded by genes, which refer to specific protein molecules directed against antigenic sites and include all polyclonal antibodies, monoclonal antibodies, recombinant antibodies, and the like.

Measurement of the expression level of the protein may be performed using quantitative and qualitative protein analysis methods known in the art. Examples of these analysis methods include, but are not limited to, enzyme-linked immunosorbent (ELISA), radioimmunoassay (RIA), sandwich assay, Western blotting, immunoprecipitation, immunohistochemical staining, fluorescence immunoassay, enzyme substrate color development, antigen-antibody aggregation, fluorescence activated cell sorter (FACS), mass spectrometry, MRM (multiple-reaction monitoring) assay, an assay employing a set of multiplexed, amine-specific, stable isotope reagents (iTRAQ, isobaric tags for relative and absolute quantitation), protein chip assay, and the like.

The composition for diagnosis of muscle weakness-related disease according to the present invention, which comprises an agent(s) for measuring the expression level of tetranectin, gelsolin, or tetranectin and gelsolin, may further comprise an agent for measuring the expression level of any one or more proteins selected from the group consisting of macrophage migration inhibitory factor (MIF), interleukin-6 (IL-6), SPARC (secreted protein acidic and rich in cysteine) and insulin-like growth factor-1 (IGF-1).

When the composition of the present invention further comprises the agent for measuring the expression level of MIF, IL-6, SPARC or IGF-1, it can more accurately diagnose muscle weakness-related disease with high specificity and sensitivity.

Macrophage migration inhibitory factor (MIF) is a dimeric polypeptide consisting of 115 amino acids and having a molecular weight of 12.5 kDa, and is a kind of lymphokine (gene sequence: NM_002415.1; amino acid sequence: NP 002406.1).

Interleukin 6 (IL-6) is B cell stimulatory 2 (BSF-2) that induces the final differentiation of B cells into antibody-producing cells, and is a glycoprotein consisting of 183 amino acids and having a molecular weight of 22 to 28 kDa. It is also a cytokine that is produced in various cells, including T lymphocytes, B lymphocytes, macrophages, fibroblasts, and the like (gene sequence: NM_000600.4; amino acid sequence: NP_000591.1).

SPARC (secreted protein acidic and rich in cysteine), also known as BM-40, is a 43 kDa protein consisting of 286 amino acids, and is also a matricellular glycoprotein which is involved in cell adhesion and movement, cell differentiation, cell proliferation, blood vessel formation, and the like (gene sequence: NM_003118.3; amino acid sequence: NP_003109.1).

Insulin-like growth factor-1 (IGF-1), also called somatomedin C, is a 7,649 Da protein consisting of 70 amino acids, and has effects on childhood growth, adult assimilation, and the like (gene sequence: NM_000618.4; amino acid sequence: NP 000609.1).

In the present invention, the expression levels of tetranectin, gelsolin, MIF, IL-6 and SPARC may be significantly higher in a sarcopenia patient group than in a normal group, and the expression level of IGF-1 protein may be significantly lower than in a sarcopenia patient group than in a normal group.

The present invention also provides a kit for diagnosis of muscle weakness-related disease, which comprises a composition for diagnosis of diagnosis of muscle weakness-related disease, the composition comprising an agent(s) for measuring the expression level of tetranectin, gelsolin, or tetranectin and gelsolin.

Specifically, the kit may be a kit for diagnosis of muscle weakness-related disease, which comprises the composition for diagnosis of muscle weakness-related disease, the composition further comprising an agent for measuring the expression level of any one or more proteins selected from the group consisting of macrophage migration inhibitory factor (MIF), interleukin-6 (IL-6), SPARC (secreted protein acidic and rich in cysteine), and insulin-like growth factor-1 (IGF-1).

In the kit for diagnosis of muscle weakness-related disease according to the present invention, the muscle weakness-related disease may be any one or more selected from the group consisting of sarcopenia, muscular atrophy, muscular dystrophy, cachexia, and acardiotrophy, but is not limited thereto. According to one embodiment of the present invention, the muscle weakness-related disease may be sarcopenia or muscular atrophy.

The kit for diagnosis of muscle weakness-related disease according to the present invention can diagnose muscle weakness-related disease by analyzing quantitatively or qualitatively analyzing the protein. Measurement of the protein may be performed using a method such as enzyme-linked immunosorbent (ELISA), radioimmunoassay (RIA), sandwich assay, Western blotting, immunoprecipitation, immunohistochemical staining, fluorescence immunoassay, enzyme substrate color development, antigen-antibody aggregation, fluorescence activated cell sorter (FACS), mass spectrometry, MRM (multiple-reaction monitoring) assay, an assay employing a set of multiplexed, amine-specific, stable isotope reagents (iTRAQ, isobaric tags for relative and absolute quantitation), protein chip assay, or the like.

For example, the kit for diagnosis according to the present invention may comprise essential elements required for performing ELISA. The ELISA kit comprises antibodies specific for the proteins. The antibodies are monoclonal antibodies, polyclonal antibodies or recombinant antibodies, which have high specificity and affinity for the marker proteins and have little or no cross-reactivity with other proteins. In addition, the ELISA kit may comprise an antibody specific for a control protein. In addition, the ELISA kit may also comprise reagents which may detect bound antibodies, for example, labeled secondary antibodies, chromophores, enzymes (e.g. conjugated with antibodies) and the substrates thereof or other substances which are capable of binding antibodies.

The present invention also provides a method for providing information for diagnosis of muscle weakness-related disease, the method comprising the steps of: (a) measuring the expression levels of tetranectin and gelsolin proteins in a biological sample obtained from a subject; and (b) determining that the subject has muscle weakness-related disease, when the expression levels of tetranectin and gelsolin proteins are higher than those in a normal group.

As used herein, the term “biological sample” in step (a) includes a sample which shows a difference in the expression of protein or gene due to muscle weakness-related disease. Specifically, the term refers to blood, serum or plasma. The biological sample may be one isolated from the human body.

Methods for measuring the expression expressions of the proteins in step (a) include, but are not limited to, enzyme-linked immunosorbent (ELISA), radioimmunoassay (RIA), sandwich assay, Western blotting, immunoprecipitation, immunohistochemical staining, fluorescence immunoassay, enzyme substrate color development, antigen-antibody aggregation, fluorescence activated cell sorter (FACS), mass spectrometry, MRM (multiple-reaction monitoring) assay, an assay employing a set of multiplexed, amine-specific, stable isotope reagents (iTRAQ, isobaric tags for relative and absolute quantitation), protein chip assay, and the like.

The “normal group” in step (b) includes groups who have not developed muscle weakness-related disease or have recovered from the disease, including a normal group having no muscle weakness-related disease, and a group who has recovered from muscle weakness-related disease and maintained muscle mass and muscle fibers at the levels shown in the normal group. According to the present invention, when the expression levels of tetranectin and gelsolin proteins are higher than those in the normal group, the subject may be classified as a patient group having muscle weakness-related disease.

The present invention also provides a method for providing information for diagnosis of muscle weakness-related disease, the method comprising the steps of: (a) measuring the expression levels of tetranectin, gelsolin, and any one or more proteins selected from the group consisting of macrophage migration inhibitory factor (MIF), interleukin-6 (IL-6), SPARC (secreted protein acidic and rich in cysteine) and insulin-like growth factor (IGF-1), in a biological sample obtained from a subject; (b) calculating a risk score based on the expression levels measured in step (a); and (c) comparing the calculated risk score with a reference level, and determining that the subject has muscle weakness-related disease, when the risk score is equal to or higher than the reference level.

In the present invention, when step (a) of measuring the expression level of MIF, IL-6, SPARC or IGF-1, in addition to the expression levels of tetranectin and gelsolin, is performed, information for diagnosing muscle weakness-related disease with significantly increased specificity and sensitivity can be provided.

Measurement of the expression levels of the proteins in step (a) of measuring the expression levels of tetranectin, gelsolin, and any one or more proteins selected from the group consisting of macrophage migration inhibitory factor (MIF), interleukin-6 (IL-6), SPARC (secreted protein acidic and rich in cysteine) and insulin-like growth factor (IGF-1), in a biological sample, is as described above.

The method for diagnosis of muscle weakness-related disease according to the present invention comprises step (b) of calculating a risk score based on the protein expression levels measured in step (a). Based on the expression levels of MIF, IL-6, SPARC or IGF-1 in addition to tetranectin and gelsolin, the risk score is calculated, thereby diagnosing the muscle weakness-related disease.

The risk score may be calculated based on a predetermined reference level value. For example, the reference level may be predetermined and set to meet routine requirements in terms of specificity, sensitivity and/or accuracy. For example, sensitivity or specificity may be set to certain limits, e.g. 60%, 70%, 80%, 90% or 95%, respectively. These requirements may also be defined in terms of positive or negative predictive values. The reference level may be predetermined in reference samples from healthy individuals (e.g., a normal group of the same age, which has no muscle weakness-related disease) or predetermined from the disease entity to which the patient belongs.

In the present invention, a statistical analysis method such as mean value calculation or ROC curve analysis may be used to determine the reference level of expression.

ROC curve analysis according to one embodiment of the present invention is performed in terms of sensitivity, specificity and accuracy using a curve that shows the performance of diagnosis.

When both specificity and sensitivity are high, the accuracy of test results increases. Thus, the x-axis in the ROC curve is 1-specificity (false positive rate), and the y-axis is sensitivity (true positive rate), and the AUC (area under curve) indicating accuracy means the area under the curve.

According to one embodiment of the present invention, “reference level” is a threshold value that shows an effect on the diagnosis of muscle weakness-related disease.

According to one embodiment of the present invention, the concentration (expression level) of each protein, measured in each of the normal group and the disease group, is log transformed, and then the linear regression coefficient corresponding to each protein is multiplied to obtain a risk score for each biomarker, and the maximum value of the product of sensitivity and specificity is determined as cut-off.

In addition, when a combination of proteins measured is applied as multiple biomarkers, the risk score of the multiple biomarkers can be normalized by correcting the risk score of each protein. Specifically, as indicated in the following Equation 1, the risk score of the multiple biomarkers can be normalized by the sum of the single marker risk scores multiplied by the linear regression coefficient corresponding to each protein.

Risk score of multiple markers=Σ logistic regression coefficient of molecule Mᵢ×log₂ transformed serum level of molecule Mᵢ Equation 1

The method for diagnosis of muscle weakness-related disease according to the present invention comprises step (c) of comparing the calculated risk score with a reference level, and determining that the subject has muscle weakness-related disease, when the risk score is equal to or higher than the reference level.

As used herein, the term “more than” or “higher than” means a level higher than the reference level or means an overall increase of 1%, 2%, 5%, 10%, 20%, 25%, 30%, 40%, 50%, 60%, 70%, 80%, 85%, 90%, 95%, 96%, 97%, 98%, 99% or more in the expression level detected by the method described herein, compared to the expression level in the reference sample. As used herein, the term “less than” or “lower than” means a level lower than the reference level or means an overall decrease of 1%, 2%, 5%, 10%, 20%, 25%, 30%, 40%, 50%, 60%, 70%, 80%, 85%, 90%, 95%, 96%, 97%, 98%, 99% or more in the expression level detected by the method described herein, compared to the expression level in the reference sample.

In the step of determining that the subject has muscle weakness-related disease, the subject from which the sample was obtained may be diagnosed to have muscle weakness-related disease, when the risk score is equal to or higher than the reference level.

### Advantageous Effects of Invention

The composition for diagnosis of muscle weakness-related disease according to the present invention makes it possible to diagnose muscle weakness-related disease in an easy and rapid manner by molecular diagnosis, thereby systemically managing the muscle weakness-related disease while increasing therapeutic efficacy against the muscle weakness-related disease.

### BRIEF DESCRIPTION OF DRAWINGS

FIG. 1 shows the results of ELISA performed to measure serum MIF, IL-6, SPARC, IGF-1, gelsolin and tetranectin levels in a normal control group and a sarcopenia patient group.

FIG. 2 depicts receiver operating characteristics (ROC) graphs showing serum MIF, IL-6, SPARC, IGF-1, gelsolin and tetranectin levels in a normal control group and a sarcopenia patient group.

FIG. 3 shows the results of ROC curve analysis performed with a combination of two biomarkers (gelsolin and tetranectin) to confirm the classification of sarcopenia patients.

FIG. 4 shows the results of ROC curve analysis performed with a combination of three biomarkers (gelsolin, tetranectin and MIF) to confirm the classification of sarcopenia patients.

FIG. 5 shows the results of ROC curve analysis performed with a combination of three biomarkers (gelsolin, tetranectin and IL-6) to confirm the classification of sarcopenia patients.

FIG. 6 shows the results of ROC curve analysis performed with a combination of three biomarkers (gelsolin, tetranectin and SPARC) to confirm the classification of sarcopenia patients.

FIG. 7 shows the results of ROC curve analysis performed with a combination of three biomarkers (gelsolin, tetranectin and IGF-1) to confirm the classification of sarcopenia patients.

FIG. 8 shows the results of ROC curve analysis performed with a combination of six biomarkers (gelsolin, tetranectin, IL-6, SPARC, MIF and IGF-1) to confirm the classification of sarcopenia patients.

FIG. 9 shows the results of ELISA performed to measure serum gelsolin and tetranectin levels in muscular atrophy mouse models.

### MODE FOR THE INVENTION

The advantages and features of the present invention, and the way of attaining them, will become apparent with reference to the examples described below. However, the present invention is not limited to the examples disclosed below and can be embodied in a variety of different forms; rather, these examples are provided so that this disclosure will be thorough and complete, and will fully convey the scope of the present invention to those skilled in the art. The scope of the present invention will be defined by the appended claims.

### Example 1. Selection of Test Subjects and Measurement of Serum Protein Levels

In order to develop a method of diagnosing muscle weakness-related disease by use of blood biomarkers, among the elderly aged 60 or older, elderly persons with normal muscle mass and elderly persons with sarcopenia were selected as test subjects. The criteria for selection of sarcopenia were set as follows:

Appendicular skeletal muscle mass(ASM)−ASM(kg)/height(m)²:male<7.0 kg/m²,female<5.7 kg/m²

To measure serum proteins, serum protein levels were measured by an ELISA technique. Specifically, R&D systems Quantikine Elisa kits (Human IL-6, Cat # D6050; Human MIF, Cat # DMF00B; Human SPARC, Cat # DSP00; and Human IGF-1, DG100) and MyBioSource Elisa kits (Gelsolin, Cat # MBS7228324; Tetranectin, Cat # MBS762655) were used, and each serum protein level was measured using the protocol provided in each of the kits, thereby determining the serum levels of tetranectin, gelsolin, MIF (macrophage migration inhibitory factor), IL-6 (interleukin 6), SPARC (secreted protein acidic and rich in cysteine), and IGF-1 (Insulin-Like Growth Factor-1).

### Example 2. Risk Score Calculation and Statistical Analysis

To compare the protein levels measured in Example 1, each protein level was log₂ transformed and subjected to Logistic regression analysis.

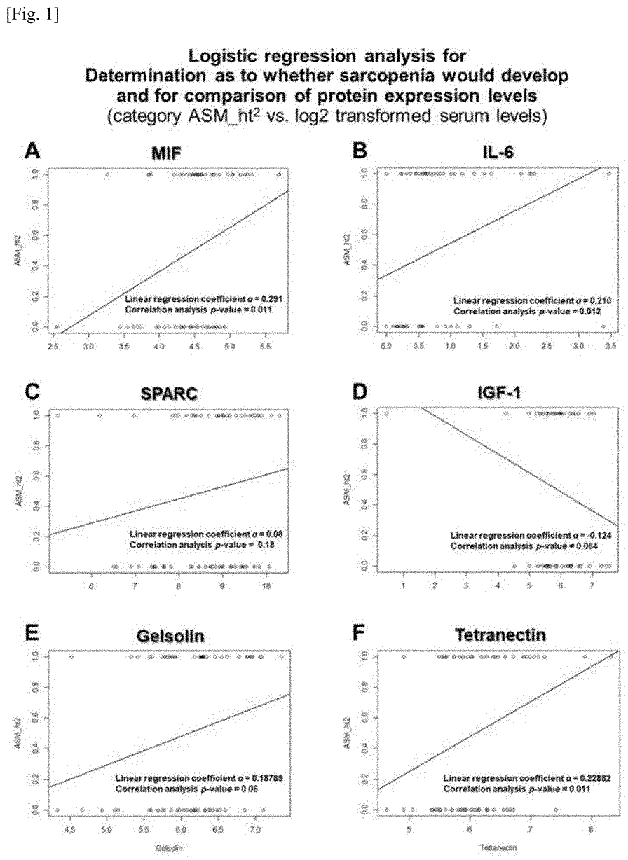

The results are shown in FIG. 1.

As can be seen in FIG. 1, the serum levels of tetranectin, gelsolin, MIF, IL-6, SPARC and IGF-1 were all significantly different between the elderly persons with normal muscle mass and the elderly persons with sarcopenia. Specifically, the serum levels of tetranectin, gelsolin, MIF, IL-6 and SPARC were higher in the elderly persons with normal muscle mass than in the patient group with sarcopenia, and the serum level of IGF-1 was higher in the elderly persons with normal muscle mass.

Furthermore, to apply the measured proteins as multiple biomarkers, correction of a sarcopenia risk score according to each of the serum protein levels was performed. Specifically, the risk score for each protein was calculated by multiplying the linear regression coefficient corresponding to each protein in order to reduce the variable between the markers. The linear regression coefficient for each protein is shown in FIG. 1. The risk score of multiple markers was defined as the sum of the risk scores of the individual markers, and was calculated using the following Equation 1:

Risk score of multiple markers=Σlogistic regression coefficient of molecule Mᵢ×log 2 transformed serum level of molecule Mᵢ Equation 1

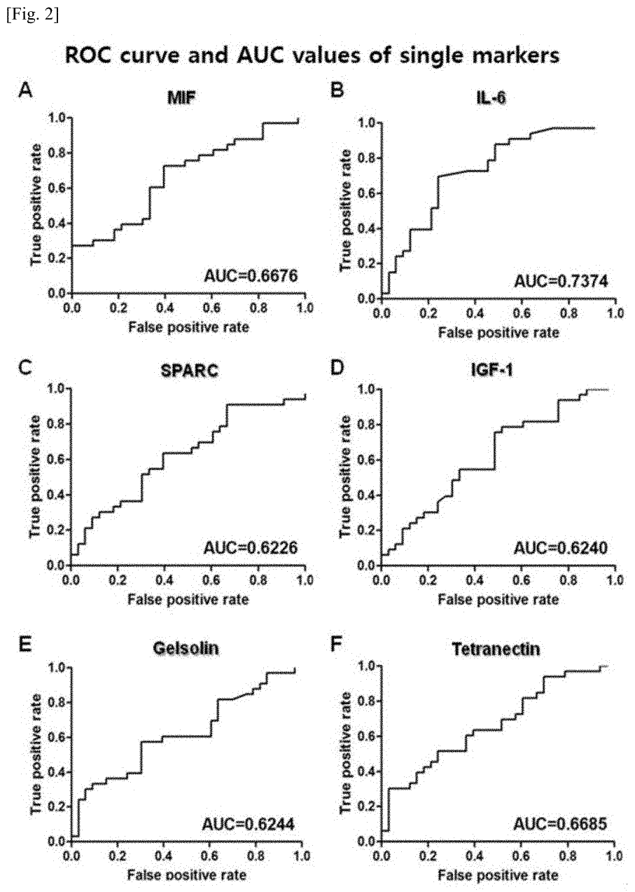

In addition, each of the protein levels was expressed as a receiver operating characteristics (ROC) graph, and the results are shown in FIG. 2.

As can be seen in FIG. 2, tetranectin, gelsolin, MIF, IL-6, SPARC and IGF-1 all had high sensitivity, specificity and AUC values, suggesting that they can be used as single markers to diagnose sarcopenia.

In addition, all statistical analyses were performed using GraphPad Prism5 (GraphPad Software, Inc., USA) and R language environment (ver. 3.2.5). The difference between the control group and the test group was statistically analyzed by two tailed, unpaired Student's t-test, and sensitivity and specificity were calculated for combinations of the biomarkers, and AUC values were calculated using ROC curves. Furthermore, maximum value of the product of sensitivity and specificity was determined as cut-off and P value<0.05 was determined statistically significant.

### Example 3. Analysis of Significance of Multiple Biomarkers for Diagnosis

(1) Analysis of Signification of Combination of Two Biomarkers (Gelsolin and Tetranectin) for Diagnosis

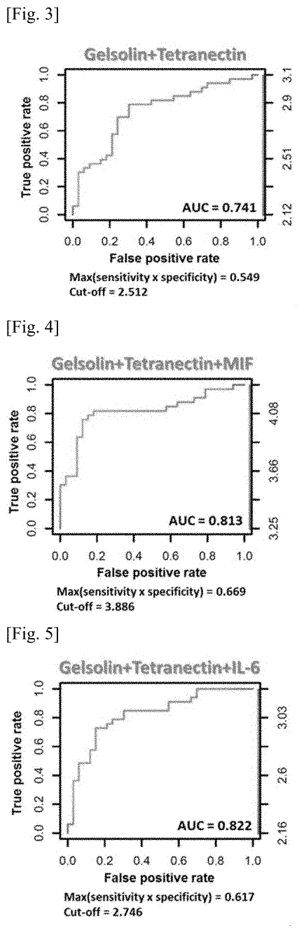

According to the method described in Example 2 above, the significance of a combination of two biomarkers (gelsolin and tetranectin) for diagnosis was analyzed. The results are shown in FIG. 3.

As shown in FIG. 3, the AUC value of the two-biomarker combination (gelsolin and tetranectin) in the elder persons with normal muscle mass and the elder persons with sarcopenia was 0.741, the maximum value of the product of sensitivity and specificity was 0.549, and the cut-off value was 2.512.

These results suggest that the two-biomarker combination (gelsolin and tetranectin) according to the present invention can diagnose sarcopenia with high accuracy.

(2) Analysis of Signification of Combination of Three Biomarkers (Including Gelsolin and Tetranectin) for Diagnosis

Analysis of Signification of Combination of Three Biomarkers (Gelsolin Tetranectin and MIF) for Diagnosis

According to the method described in Example 2 above, the significance of a combination of three biomarkers (gelsolin tetranectin and MIF) for diagnosis was analyzed. The results are shown in FIG. 4.

As shown in FIG. 4, the AUC value of the three-biomarker combination (gelsolin, tetranectin and MIF) in the elder persons with normal muscle mass and the elder persons with sarcopenia was 0.813, the maximum value of the product of sensitivity and specificity was 0.669, and the cut-off value was 3.886.

Analysis of Signification of Combination of Three Biomarkers (Gelsolin, Tetranectin and IL-6) for Diagnosis

According to the method described in Example 2 above, the significance of a combination of three biomarkers (gelsolin tetranectin and IL-6) for diagnosis was analyzed. The results are shown in FIG. 5.

As shown in FIG. 5, the AUC value of the three-biomarker combination (gelsolin, tetranectin and IL-6) in the elder persons with normal muscle mass and the elder persons with sarcopenia was 0.822, the maximum value of the product of sensitivity and specificity was 0.617, and the cut-off value was 2.746.

Analysis of Signification of Combination of Three Biomarkers (Gelsolin, Tetranectin and SPARC) for Diagnosis

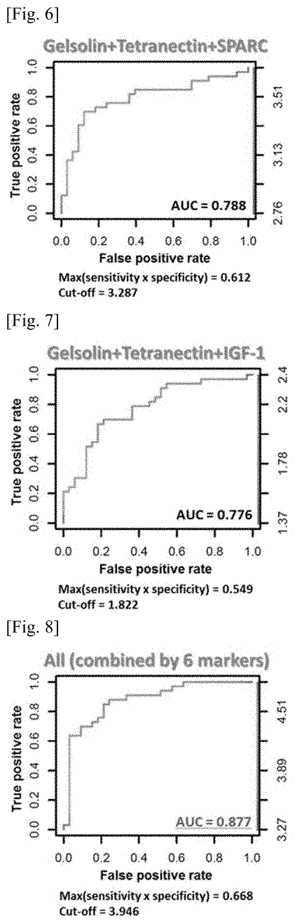

According to the method described in Example 2 above, the significance of a combination of three biomarkers (gelsolin tetranectin and SPARC) for diagnosis was analyzed. The results are shown in FIG. 6.

As shown in FIG. 6, the AUC value of the three-biomarker combination (gelsolin, tetranectin and IL-6) in the elder persons with normal muscle mass and the elder persons with sarcopenia was 0.788, the maximum value of the product of sensitivity and specificity was 0.612, and the cut-off value was 3.287.

Analysis of Signification of Combination of Three Biomarkers (Gelsolin, Tetranectin and IGF-1) for Diagnosis

According to the method described in Example 2 above, the significance of a combination of three biomarkers (gelsolin tetranectin and SPARC) for diagnosis was analyzed. The results are shown in FIG. 7.

As shown in FIG. 7, the AUC value of the three-biomarker combination (gelsolin, tetranectin and IGF-1) in the elder persons with normal muscle mass and the elder persons with sarcopenia was 0.776, the maximum value of the product of sensitivity and specificity was 0.549, and the cut-off value was 1.822.

These results indicate that the three-biomarker combination including MIF, IL-6, SPARC or IGF-1 together with gelsolin and tetranectin shows a significantly increased AUC value compared to the two-biomarker combination of gelsolin and tetranectin.

(3) Analysis of Significance of Multiple Biomarkers (Gelsolin, Tetranectin, IL-6, MIF, SPARC and IGF-1) for Diagnosis

According to the method described in Example 2 above, the significance of multiple biomarkers (gelsolin, tetranectin IL-6, MIF, SPARC and IGF-1) for diagnosis was analyzed. The results are shown in FIG. 8.

As shown in FIG. 8, the total AUC value of gelsolin, tetranectin IL-6, MIF, SPARC and IGF-1 in the elder persons with normal muscle mass and the elder persons with sarcopenia was 0.877, which was the highest AUC value. In addition, the maximum value of the product of sensitivity and specificity was 0.668, and the cut-off value was 3.946.

From these results, it was found that the multiple biomarkers including gelsolin and tetranectin according to the present invention all had high sensitivity, specificity and AUC values, suggesting that they can be used as diagnostic biomarkers to detect sarcopenia.

### Example 4. Construction of Muscle Atrophy Mouse Models and Analysis of Markers

To induce muscle atrophy in C57BL/6J male mice (the Laboratory Animal Resource Center at the Korea Research Institute of Bioscience and Biotechnology), the TA (tibialis anterior) muscle immobilization method was used (Caron A Z, J Appl Physiol. 106(6) 2049-2059, 2009). The principle used in this method is that when a leg is placed in a cast and the muscle of the leg is immobilized (i.e., not frequently used), the muscle is lost. It is a method for inducing muscle regeneration, in which, after the muscle loss due to immobilizing the shin muscle, the muscle is regenerated by releasing the immobilized muscle so that the muscle may move again. Specifically, the thighs and shins of both legs of mice were fixed using a medical staple so that legs were immobilized, and after leaving the immobilized mice alone for 5 days, the fixed legs were released, thereby constructing muscle atrophy mouse models.

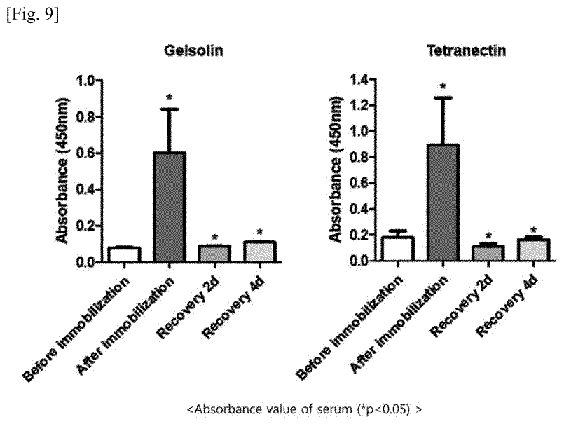

In order to measure the change in serum protein levels by the induction of muscle atrophy, serum was isolated from the mice before immobilization of the mouse legs, on 5 days after immobilization, and on 2 and 4 days after release of the mouse legs, and the levels of tetranectin and gelsolin in the serum were analyzed using MyBioSource Elisa kits (Gelsolin, Cat # MBS2886136; Tetranectin, Cat # MBS2885296). The results are shown in FIG. 9.

As shown in FIG. 9, when muscle atrophy was induced by immobilization of the legs, the serum tetranectin and gelsolin levels all significantly increased compared to those before immobilization and during the recovery period (2 days and 4 days after release of the legs). These results indicate that tetranectin and gelsolin according to the present invention may be used as markers to detect muscle atrophy.

The above-described results suggest that muscle weakness-related disease can be effectively diagnosed with high accuracy by measuring tetranectin, gelsolin, or blood biomarkers including them.

1-16. (canceled)

17. A kit for diagnosis of muscle weakness-related disease, which comprises:
(a) an agent for measuring an expression level of gelsolin in a biological sample; and/or
(b) an agent for measuring an expression level of tetranectin in the biological sample,
wherein the (a) agent and/or the (b) agent contain a label which can be detected by any one selected from the group consisting of enzyme-linked immunosorbent (ELISA), radioimmunoassay (RIA), sandwich assay, Western blotting, immunoprecipitation, immunohistochemical staining, fluorescence immunoassay, enzyme substrate color development, antigen-antibody aggregation, fluorescence activated cell sorter (FACS), mass spectrometry, MRM (multiple-reaction monitoring) assay, an assay employing a set of multiplexed, amine-specific, stable isotope reagents (iTRAQ, isobaric tags for relative and absolute quantitation), and protein chip assay.

18. The kit of claim 17, which further comprises (c) an agent for measuring any one or more proteins selected from the group consisting of macrophage migration inhibitory factor (MIF), interleukin-6 (IL-6), SPARC (secreted protein acidic and rich in cysteine), and insulin-like growth factor-1 (IGF-1) in the biological sample.

19. The kit of claim 17, wherein the muscle weakness-related disease is any one or more selected from the group consisting of sarcopenia, muscular atrophy, muscular dystrophy, cachexia, and acardiotrophy.

20. The kit of claim 17, wherein the (a) agent is a labeled antibody which specifically binds gelsolin, and/or the (b) agent is a labeled antibody which specifically binds to tetranectin.

21. The kit of claim 18, wherein the (a) agent is a labeled antibody which specifically binds gelsolin, and/or the (b) agent is a labeled antibody which specifically binds to tetranectin.

22. The kit of claim 19, wherein the (a) agent is a labeled antibody which specifically binds gelsolin, and/or the (b) agent is a labeled antibody which specifically binds to tetranectin.

23. The kit of claim 18, wherein the (c) agent is a labeled antibody which specifically binds to any one or more proteins selected from the group consisting of macrophage migration inhibitory factor (MIF), interleukin-6 (IL-6), SPARC (secreted protein acidic and rich in cysteine), and insulin-like growth factor-1 (IGF-1) in the biological sample.

24. The kit of claim 23, wherein the (c) agent comprises one or more selected from the group consisting of:
(c-1) a labeled antibody specifically binding to macrophage migration inhibitory factor (MIF),
(c-2) a labeled antibody specifically binding to interleukin-6 (IL-6),
(c-3) a labeled antibody specifically binding to SPARC (secreted protein acidic and rich in cysteine), and
(c-4) a labeled antibody specifically binding to insulin-like growth factor-1 (IGF-1).

25. A method for providing information for diagnosis of muscle weakness-related disease of a subject, the method comprising the steps of:
(i) measuring expression levels of tetranectin and/or gelsolin proteins in a biological sample obtained from the subject; and
(ii) determining that the subject has muscle weakness-related disease, when the expression levels of the tetranectin and/or gelsolin proteins are higher than reference expression levels of a control subject free of muscle weakness-related disease,
wherein the (i) is carried out using (a) an agent for measuring the expression level of gelsolin and/or (b) an agent for measuring the expression level of tetranectin,
wherein the (a) agent and/or the (b) agent contain a label which can be detected by any one selected from the group consisting of enzyme-linked immunosorbent (ELISA), radioimmunoassay (RIA), sandwich assay, Western blotting, immunoprecipitation, immunohistochemical staining, fluorescence immunoassay, enzyme substrate color development, antigen-antibody aggregation, fluorescence activated cell sorter (FACS), mass spectrometry, MRM (multiple-reaction monitoring) assay, an assay employing a set of multiplexed, amine-specific, stable isotope reagents (iTRAQ, isobaric tags for relative and absolute quantitation), and protein chip assay.

26. The method of claim 25, wherein the biological sample in step (a) is any one selected from the group consisting of blood, serum, and plasma of the subject.

27. The method of claim 25, wherein, in (ii), the expression level of the gelsolin and/or the expression level of tetranectin of the subject are higher than about 10% of the reference levels, respectively, indicate that the subject has muscle weakness-related disease.

28. The method of claim 25, further comprises
(iii) measuring expression level of any one or more proteins selected from the group consisting of macrophage migration inhibitory factor (MIF), interleukin-6 (IL-6), SPARC (secreted protein acidic and rich in cysteine) and insulin-like growth factor-1 (IGF-1), in the biological sample,
wherein the (iii) is carried out using (c) an agent for measuring the expression level of the any one or more proteins selected from the group consisting of macrophage migration inhibitory factor (MIF), interleukin-6 (IL-6), SPARC (secreted protein acidic and rich in cysteine), and insulin-like growth factor-1 (IGF-1), and
wherein the (c) agent contains a label which can be detected by any one selected from the group consisting of enzyme-linked immunosorbent (ELISA), radioimmunoassay (RIA), sandwich assay, Western blotting, immunoprecipitation, immunohistochemical staining, fluorescence immunoassay, enzyme substrate color development, antigen-antibody aggregation, fluorescence activated cell sorter (FACS), mass spectrometry, MRM (multiple-reaction monitoring) assay, an assay employing a set of multiplexed, amine-specific, stable isotope reagents (iTRAQ, isobaric tags for relative and absolute quantitation), and protein chip assay.

29. The method of claim 28, further comprises
(iv) determining that the subject has muscle weakness-related disease, when the expression levels of the any one or more proteins selected from the group consisting of macrophage migration inhibitory factor (MIF), interleukin-6 (IL-6), SPARC (secreted protein acidic and rich in cysteine), and insulin-like growth factor-1 (IGF-1) is higher than reference expression level of the control subject.

30. The method of claim 28, wherein, in (iv), the expression level of the gelsolin and the expression level of tetranectin of the subject are higher than about 10%/o of the reference levels, respectively, indicate that the subject has muscle weakness-related disease.

31. The method of claim 28, wherein the (i) and (iii) are carried out as a single test.

32. The method of claim 28, wherein the (c) agent comprises one or more selected from the group consisting of:
(c-1) a labeled antibody specifically binding to macrophage migration inhibitory factor (MIF),
(c-2) a labeled antibody specifically binding to interleukin-6 (IL-6),
(c-3) a labeled antibody specifically binding to SPARC (secreted protein acidic and rich in cysteine), and
(c-4) a labeled antibody specifically binding to insulin-like growth factor-1 (IGF-1).
